(19) United States
(12) Patent Application Publication
(10) Pub. No.: US 2014/0069857 A1
(43) Pub. Date: Mar. 13, 2014

(54) PRESSURE CONVERTER-PROTECTIVE DEVICE FOR BLOOD TUBING SYSTEMS

(71) Applicant: Fresenius Medical Care Deutschland GmbH, Bad Homburg (DE)

(72) Inventor: Christoph BRUECKNER, Geldersheim (DE)

(73) Assignee: Fresenius Medical Care Deutschland GmbH, Bad Homburg (DE)

(21) Appl. No.: 14/026,770

(22) Filed: Sep. 13, 2013

Related U.S. Application Data
(60) Provisional application No. 61/700,549, filed on Sep. 13, 2012.

(30) Foreign Application Priority Data
Sep. 13, 2012 (DE) 102012018078.0

(51) Int. Cl. A61M 1/16

(52) U.S. Cl. CPC A61M 1/1656
USPC 210/232

(57) ABSTRACT

The invention relates to a protective device for a pressure converter in a blood treatment machine. Traditional converter-protective devices are not easy to mount on the treatment machine because they offer the user hardly any gripping surface. With the converter-protective device according to the present invention, the housing with the anti-contamination barrier can be gripped on the main surface in mounting it and can be inserted into the machine. This is achieved by the fact that the connector axes and the housing with the anti-contamination barrier are situated in parallel planes which are arranged perpendicular to the surface of the device after assembly. Due to this orientation, both sides of the anti-contamination barrier can be observed from the front at the sides during operation so that rupture of the anti-contamination barrier can easily be detected by the user.

### SUMMARY

The invention relates to the field of protective devices for pressure converters and machines for extracorporeal treatment of blood such as dialysis machines.

### PRIOR ART

Protective devices for pressure converters of dialysis machines are known from EP 0 878 628 B1, EP 0 652 018 B1 and EP 2 253 343 A1, for example.

### STATEMENT OF OBJECT

In methods for extracorporeal treatment of blood, e.g., hemodialysis, the patient's blood is transported from a treatment machine through a blood tubing system with a treatment medium, e.g., a dialyzer. In this extracorporeal blood circulation, the pressure must be monitored in order to maintain surveillance of physiological and procedural parameters in the extracorporeal system to detect any obstructions in the extracorporeal system or patient access problems, for example. To monitor the pressure in the extracorporeal circulation, machines for extracorporeal blood treatment have a pressure converter connected to the blood tubing system. To prevent contamination of this device by patient blood on the one hand and to ensure the stability of the blood circulation on the other hand, a gas-permeable anti-contamination barrier must be provided between the treatment machine and the extracorporeal blood circulation. The converter-protective devices normally used for this purpose consist of a two-part plastic housing. They have two tubular connection parts arranged coaxially. The first connecting part serves to connect the protective device to the pressure converter on the machine end. The second connecting part is connected to the blood tubing system. The two connecting parts additionally have a radial flange by means of which the two parts are permanently and tightly joined together. Between these flanges there is a gas-permeable membrane, which is arranged transversely between the tubular connections.

For attaching the converter-protective device to the dialysis machine, the nursing personnel must screw the housing together with the first connecting part into the connection on the surface of the machine that is connected to the pressure converter. To do so, the housing must be gripped by the narrow edge of this flange and screwed into the connection to the pressure converter in such a way that it is aligned with the flange, so that it is exactly parallel to the surface of the machine housing. Only a small gripping area is available and at the same time the housing is still connected to the blood tubing system on one end, which results in commercial converter-protective devices being difficult to handle, and the attachment is quite inconvenient for the user.

Another problem with conventional converter-protective devices is rapid and easy detection of error states.

If a treatment is proceeding without any problems, a sufficiently high column of air is always present between the blood level in the tubing system and the converter-protective device. However, error states may lead to an increase in the blood level, which is why protective devices are also provided. Usually hydrophobic membranes are used as the anti-contamination barrier. These membranes are permeable for gas, but blood cannot pass through them, so they can prevent blood from entering the dialysis machine to a certain extent. If the blood level then rises up to the membrane and if the resulting pressure exceeds the limit value, rupture of the membrane may occur. Then blood may enter the dialysis machine a short time later.

It is therefore important to detect the penetration of blood into the converter-protective device and in particular to detect a rupture of the membrane as quickly as possible.

Only one side of the converter-protective device is visible to the user during treatment because the area of the anti-contamination barrier of commercial converter-protective devices is perpendicular to the tubular connections and thus is also arranged parallel to the housing surface after being attached to the treatment machine.

This arrangement (the housing surface on the blood side faces the user, the housing surface away from the blood side is facing the device) prevents rapid detection of a defective membrane with leakage of blood (detectable mainly from the side facing away from the blood).

The object of the present invention is to make available a converter-protective device which is easier to mount on the dialysis machine.

In addition, detection of an imminent membrane rupture or a previous rupture is to be simplified.

According to the teaching of the present invention, this object is achieved by a converter-protective device according to claim 1, a blood tubing system according to claim 12 and an arrangement according to claim 13. Advantageous embodiments are the subject matter of the dependent claims.

### SUMMARY OF THE INVENTION

The converter-protective device according to the present invention consists of a flat transparent housing with a front wall, a rear wall and at least one side wall. The front wall and rear wall are preferably designed identically and interchangeably.

The main surface areas of the housing are formed by the front wall and the rear wall. The area is usually between 1 cm² and 5 cm². The side wall has a smaller area in comparison with the front wall and the rear wall. The distance between the front wall and the rear wall may be between 0.2 cm and 1 cm, preferably 0.5 to 0.8 cm.

If the front wall and the rear wall are designed to be round, for example, they are connected by a circumferential side wall. If the front wall and the rear wall are designed as rectangles, for example, then they are connected by four side walls. The front wall and rear wall may have any shape.

The housing may preferably be formed from two parts which are joined to one another in an airtight seal with respect to the environment and with a liquid-tight seal between the halves because of the separation by a hydrophobic membrane.

In addition, the housing has a gas-permeable liquid barrier, which divides the housing into two chambers.

The gas-permeable liquid barrier may preferably be arranged between the two parts of the housing that are connected to one another in a fluid-tight manner.

The gas-permeable liquid barrier serves to protect the pressure converter in the dialysis machine from penetration of fluid out of the tubing system during operation.

The fluid may be blood, plasma or electrolyte solutions which are carried in the tubing system.

The fluid may preferably also ensure the sterility of the tubing system after being removed from the package and can thus prevent contamination of the liquid, preferably blood.

The gas-permeable liquid barrier may be formed by any material which is permeable for gas and impermeable for liquids. The gas-permeable liquid barrier may preferably be formed by a hydrophobic membrane. In addition, the housing may have structures which serve to support the hydrophobic membrane.

The converter-protective device also comprises a first tubular connector for connection to a blood treatment machine and a second tubular connector for connection to a blood tubing system. A tubular connector is connected to each part of the housing. The two tubular connectors are positioned on the side wall of the flat housing, so that the tubular connector and the main area are oriented longitudinally, so that the front wall and the rear wall of the housing may be used as a gripping area for mounting on the blood treatment machine and are visible from the front laterally after mounting of the front wall and the rear wall.

It may be especially advantageous if the two tubular connectors are situated on opposite sides of the housing.

The two tubular connectors may be designed according to the Luer-Lock system.

The first tubular connector may preferably be designed according to the Luer-Lock system in which it may be a Luer-Lock connector of the female type. In an especially preferred embodiment, the conical connector has an outside thread.

In an alternative embodiment, the second tubular connector may also be embodied as a tube seat, to which the blood tubing system can then be glued securely.

The invention also relates to an extracorporeal blood tubing system having at least one converter-protective device according to claims 1 to 11.

The invention also relates to an arrangement of a blood treatment machine with an extracorporeal blood tubing system according to claim 12.

An exemplary embodiment is illustrated in the following figures:

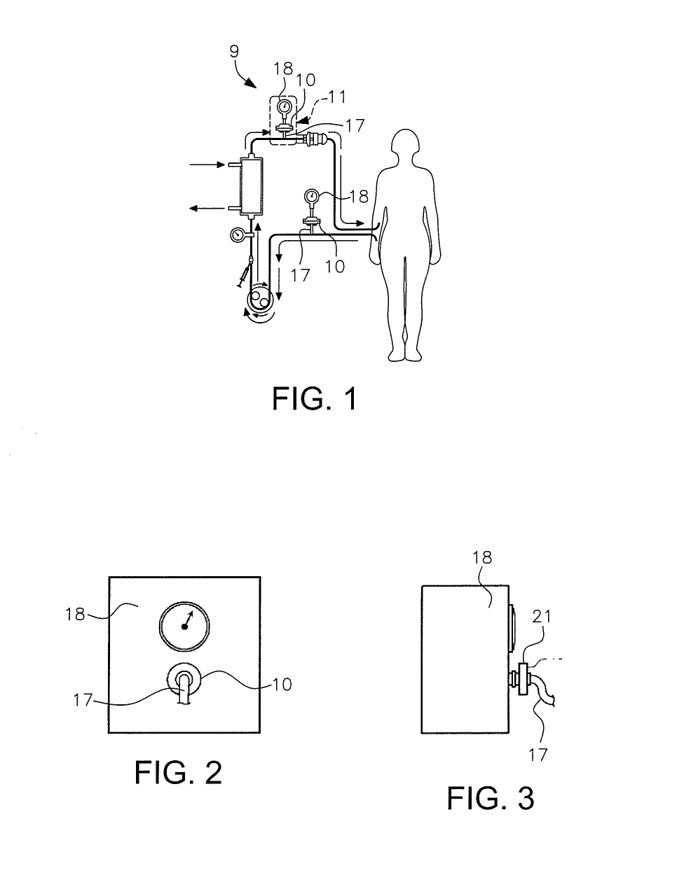

FIG. 1 shows schematically an extracorporeal circulation for a therapeutic treatment according to the prior art.

FIG. 2 shows schematically a front view of a converter-protective device according to the prior art, mounted on a blood treatment machine.

FIG. 3 shows schematically the side view of a converter-protective device according to the prior art, mounted on a blood treatment machine.

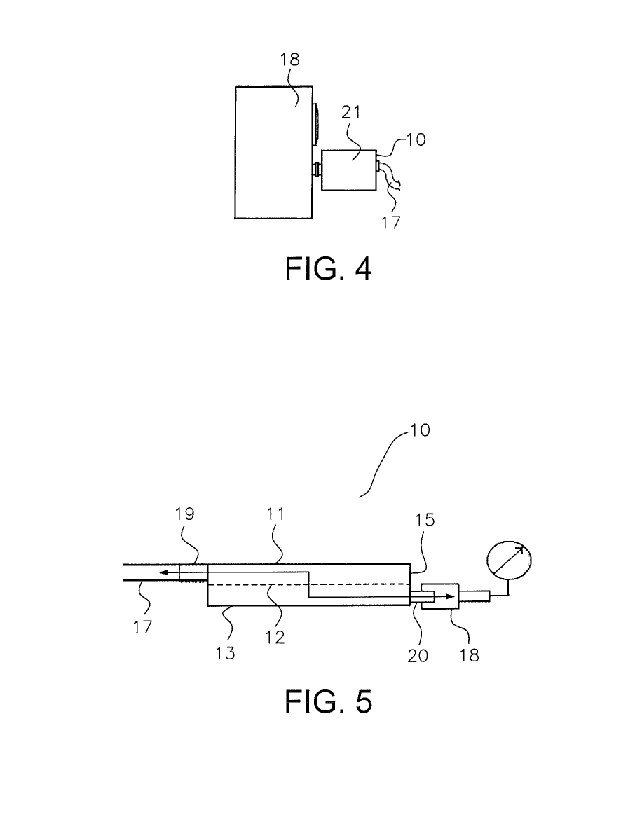

FIG. 4 shows schematically the side view of a converter-protective device according to the present invention, mounted on a blood treatment machine.

FIG. 5 shows schematically the longitudinal section through a converter-protective device according to the present invention.

### DESCRIPTION

FIG. 1 shows schematically an extracorporeal blood circulation 9 in which a pressure converter-protective device 10 is provided in both the venous branch and in the arterial branch and is connected to a pressure converter 18 of a blood treatment machine.

FIGS. 2 and 3 show a pressure converter-protective device 10 according to the prior art. After being mounted on the blood treatment machine, only the front side of the pressure converter-protective device can be seen from the front, and part of this front side is covered by the blood tubing line. Since the rear side of the device faces the machine, it is difficult for the user to detect a rupture in the liquid barrier in the protective device. In the side view in FIG. 3, the narrow gripping area 21 of this pressure converter-protective device can be seen.

FIG. 4 shows a pressure converter-protective device 10 according to the present invention, where both sides can be seen laterally from the front. This offers a large gripping area 21 by means of which the pressure converter-protective device can easily be mounted on the blood treatment machine.

FIG. 5 shows schematically a longitudinal section through a pressure converter-protective device according to the present invention. The gas-permeable liquid barrier 12 is arranged between the front wall 11 and the rear wall 13. The gas-permeable liquid barrier 12 may additionally be stabilized by supporting structures, e.g., webs (not shown). The tubular connectors 19 and 20 are arranged on the side wall 15. The tubular connector 19 is connected to the blood tubing system 17, while the tubular connector 20 is connected to the pressure converter 18 and the blood treatment machine.

1. A converter-protective device comprising a flat transparent housing consisting of a front wall and a rear wall which form the main area of the housing, at least one narrow side wall and a gas-permeable liquid barrier which divides the housing into two chambers; the converter-protective device additionally comprises a first tubular connector for connection to a blood treatment machine and a second tubular connector for connection to a blood tubing system such that a tubular connector is connected to each chamber, characterized in the tubular connector is positioned on the side wall of the flat housing, the tubular connector and the main area of the housing thereby being aligned essentially in parallel along an axis, so that the front and rear walls of the housing can be used as a gripping surface, for mounting on the blood treatment machine.

2. The device according to claim 1 characterized in that the housing consists of a first part and a second part, the two parts being connected to one another in a liquid-tight manner with the gas-permeable liquid barrier being arranged between the parts.

3. The device according to claim 1, characterized in that the gas-permeable liquid barrier is a hydrophobic membrane.

4. The device according to claim 1, characterized in that the housing has a structure for supporting the hydrophobic membrane.

5. The device according to claim 1, characterized in that the two tubular connectors are on opposite sides of the housing.

6. The device according to claim 1, characterized in that a tubular connector is arranged on each part of the housing.

7. The device according to claim 1, characterized in that one tubular connector is designed according to the Luer-Lock system.

8. The device according to claim 1, characterized in that both of the tubular connectors are designed according to the Luer-Lock system.

9. The device according to claim 1, characterized in that one tubular connector has a tube seating.

10. The device according to claim 1, characterized in that after mounting the converter-protective device on the blood treatment machine, the surface of same is oriented transversely to the longitudinal axis of the converter-protective device.

11. The device according to claim 1, characterized in that the housing is made of plastic, for example, polypropylene, polycarbonate, PVC or polysulfone.

12. An extracorporeal blood tubing system having at least one converter-protective device according to claim 1.

13. An arrangement of a blood treatment machine having an extracorporeal blood tubing system according to claim 12.
